(19) United States
(12) Patent Application Publication
(10) Pub. No.: US 2016/0017402 A1
(43) Pub. Date: Jan. 21, 2016

(54) Method and apparatus utilizing enzyme substrates producing slow diffusing fluorescent product appearances and chromogens, and use in combination with fast diffusing product appearances

(71) Applicants: Geoffrey N. Roth, Goshen, IN (US); Jonathan N. Roth, Goshen, IN (US)

(72) Inventors: Geoffrey N. Roth, Goshen, IN (US); Jonathan N. Roth, Goshen, IN (US)

(21) Appl. No.: 14/120,966

(22) Filed: Jul. 15, 2014

(51) Int. Cl. C12Q 1/25

(52) U.S. Cl. CPC C12Q 1/25

(57) ABSTRACT

A new method of rapid detection of cells, microorganisms, or other items is described using various combinations of indicator enzyme substrates which can be used to yield fluorophoric and chromophoric appearances due to enzymatic activity. One aspect of the invention is the use of a family of compounds that can be used to produce slow diffusing fluorophoric appearances. Another aspect is a family of compounds identified as dual enzyme substrates that can be used to produce both fluorophoric and chromogenic appearances.

### CROSS REFERENCE TO RELATED APPLICATIONS

This application is a continuation in part of U.S. patent application Ser. No. 13/199,419, filed on Aug. 30, 2011, which is a continuation in part of U.S. patent application Ser. No. 11/725,088, issued on Aug. 30, 2011 as U.S. Pat. No. 8,008,059. To the extent that the information herein differs substantively from the information presented in the parent patent application(s), that is the result of differences in the knowledge and/or understanding of that information and/or its applicability to the present invention between the dates of the respective application filings.

### BACKGROUND OF THE INVENTION

Current biological assays, including some microbial tests, are often non-quantitative in nature, which may pose definite problems in certain applications. Notable problems are the slowness of achieving results, and the high cost and limited availability of certain reagents used in various test formulations.

It is thought that indicator enzyme substrates (in this disclosure may be referred to as indicator substrates or enzyme substrates) have been used in methods to detect enzyme reaction or other reaction in some such assays. Substrates so used can vary with the various assays to which they are applied, some being water based. Some substrates (e.g., those known in the art as chromogens or chromogenic enzyme substrates or luminogenic enzyme substrates) can be used to allow, if reaction with the right kind of enzyme occurs, creation of a quality which may be detected, e.g., color, wavelength and/or electric energy. Herein, such a chromogenic (in this context meaning “colored”) quality may be detected using energy comprising energy from the visible light spectrum.

In addition, certain prior writers have used the term “substrates” to have other meanings. For example, Manafi has used “substrate” to describe agar.

Forms of terms “produce, produced, provide, or provide for” in this document can mean “allow at least in part due to enzyme reaction” in appropriate context of said qualities. “Produce” in this document can have a meaning chosen from a group comprising “can be used to produce” and “used to produce” and “created through the use of” in context of said qualities. “Enzyme reaction” may refer to enzyme activity of one or more molecules or units of the right kind of active enzyme, e.g., β-galactosidase, with the right kind of substrate, e.g., indoxyl-b-d-galactoside. Herein, fluorescence or a fluorescent quality is often detected using energy comprising energy from the ultraviolet spectrum.

As another example, some compounds (enzyme substrates) (e.g., those known in the art as fluorogens or fluorogenic enzyme substrates, such as, Methylumbelliferyl-B-D-galactoside, for example, can be used to allow, if reaction with the right kind of active enzyme occurs, creation of a quality that may be detected, e.g., color, wavelength and/or electric energy. Herein, such a said quality may be detected using energy which may comprise energy from the ultraviolet light spectrum.

Some enzyme substrates may need the action of more than one type of enzyme to produce a detectable quality and may be used to detect the enzymes.

Above-described qualities that can be detected, and the methods of detection of the qualities, can be referred to herein by using forms of terms including, but not limited to: detections, detected quality, detected appearances, product appearances, color, fluorescence, quality and qualities.

In the art prior to this and prior to the original disclosure, relatively few choices of types of qualities produced by enzyme substrates were used based on choice of apparent diffusiveness of the qualities in an area. One type of enzyme substrate, e.g., MUgluc (MU glucuronide), can be used to provide a type of detectable quality that, in many solid tests, e.g., in many agar and solid or broth membrane filter media, is similar, for their duration, to what Manafi describes. “The disadvantage of incorporating MUG (Mugluc) in agar is that fluorescence diffuses rapidly into the surrounding agar substrate.” (Manafi, 1991) Methylumbelliferyl-B-D-galactoside, ortho-nitrophenyl-galactoside, and some other substrates are examples of enzyme substrates that can be used to provide qualities that generally appear to rapidly diffuse in a solid test. When describing detectable qualities produced at least in part by enzyme activity, the type of qualities described in this paragraph are often referred to in this disclosure as “fast diffusing” or “fast”. This type of quality is fast for the duration of its presence with no regard of its intensity.

A second type of enzyme substrate can be used to provide a quality similar to what one generally expects in detecting a product or products from a group comprising products that, for their duration, mostly precipitate in aqueous solution, and products that for their duration diffuse slowly but remain generally localized in the vicinity of the item they are provided by, and products that for their duration can provide qualities that can mimic preferred qualities of products, according to Brenner where she teaches. “Upon cleavage, chromogens should produce insoluble or only slightly soluble chromophores that will remain localized in the bacterial colonies. Similarly, fluorophores should not diffuse away from the colonies, as excessive diffusion hinders target colony recognition, discrimination, and enumeration.” (Brenner patents, U.S. Pat. No. 6,063,590). However, choice of use of, for instance, N-methyl-3-indoxyl, 3-indoxyl, or 5-b,-4-cl-3-indoxyl based enzyme substrates, (defined below), typically allows a range in apparent diffusiveness of the qualities that the substrates can be used to produce, but the qualities still do not for the most part appear fast and so can be used to localize enzyme activity. This type of quality may seem to stain the entity that it can be used to detect. 6-cl-3-indolyl-B-D-galactoside, and napthol-B-D-galactoside used with diazonium salts are examples of such enzyme substrates that can be used to provide such a quality. This type of quality may not appear to diffuse much in broth, but with a solid assay (e.g.—in an agar or membrane filter microbial assays), it can be used to permit one colony (C.F.U.—colony forming unit) or more than one colony to be detected and enumerated. This may not be possible in the case of the presence of too many items (e.g.—colonies) to be separately detected. When describing detectable qualities produced at least in part by enzyme activity, the type of quality described in this paragraph is often referred to in this disclosure as “slow diffusing” or “slow”. This type of quality is slow for the duration of its presence regardless of its intensity. There may also be enzyme substrates that can be used to provide for intermediate qualities, but it is thought they tend toward either slow or fast. The use of some enzyme substrates produces qualities that appear to be affected by pH. Such substrates have been referred to as “pH-fluorescent indicators” in the art. One example is Mugluc. (MUG glucuronide, methyl umbelliferyl β-D-glucuronide).

Many examples herein describing use of an enzyme substrate used to produce detectable qualities may use forms of the terms chosen from a group including but not limited to the following: produce, fast, slow, fluorescent, fluorgenic, chromogenic, color or quality. For instance “producing slow fluorogenic qualities” uses forms of these terms. “Definitions” set forth at the end of this specification are to be understood in light of the above explanations.

In recent years the use of chromogenic, fluorogenic and other enzyme indicators has grown in favor for certain applications due to ease of use in enzyme reaction assays for food, water, cancer, disease, tissue, enzyme and microbe research, and commerce in the biological area. Edberg patented a broth with a substrate that can be used to produce a fast fluorescence and a substrate that can be used to produce a fast chromogenic appearance. Brenner (previous citation) essentially teaches that substrates used to produce fast qualities are inferior as the qualities that the substrates can be used to produce may diffuse from the items they identify. The use of indoxyl based substrates to produce a slow chromogenic quality has been an alternative. Ley (U.S. Pat. No. 4,923,804) received a patent involving the use of indoxyl-B-glucuronide to detect E. coli with membrane filtration. Ferguson (U.S. Pat. No. 5,358,854) synthesized a novel chloro-indoxyl based substrate for Roth to use to detect coliforms, and Roth combined this with a bromo-chloro-indoxyl based substrate to use to detect and differentiate E. coli and other coliforms. Roth used enzyme reactions with two different chromogenic indoxyl based substrates, to detect two different enzymes at once.

It is thought that indoxyl based substrates have been generally commercially available worldwide and generally accepted as non-toxic substrates that, due to enzyme reaction, provide for non-toxic detectable products. Indoxyl or indoxyl with substitutions, (eg—6-cl-indoxyl) based substrates have been recommended for use by the Biosynth company, Ley, Manafi, Ferguson, Roth, Brenner, Dufour (by product offerings, inclusion in patents and approvals for media given) and others. This is also generally true of substrates that can be used to produce nitrophenol or methylumbelliferone.

Other substrates are known in histochemistry. Haugland (U.S. Pat. Nos. 5,316,906 and 5,443,986) teaches immuno-histochemical detection of enzyme activity using one of “substrates made from a class of fluorophores, generally including quinazolinones (quinazolones), benzimidazoles, benzothiazoles, benzoxazoles, quinolines, indolines, and phenanthridines . . . ”. He uses a substrate, 2-(5′-chloro-2′phosphoryloxyphenyl)-6-chloroquinazolinone to detect enzyme activity. The related phosphatase substrate 2-(5′-chloro-2′-phosphoryloxyphenyl)-4-[³]-quinazolinone is referred to as “CPQP” and “ELF-97” is an analogue. (van Ommen Klocke, 1999). CPQP is used with a streak plate method and was added as a supplement after the medium has been sterilized, specifically with streaked agar methods in microbial assay art. (van Ommen Klocke, 1999, Wick, WO 2009/061733 A2, US 2010/0291591 A1). This may not be an ideal test because streak plate methods may not be very quantitative. A pour plate or membrane filtration test are often more accurately quantitative and preferred.

The above teaching was evident prior to the original disclosure of the invention to which this is a second continuation in part.

It is thought that indoxyl (indoxyl, indol-3-yl, 3-indolyl, indolyl, and 1H-indol-3-ol are synonymous where “3” indicates the position of oxygen on the carbon at the “3” position), (Ferguson, personal communication, Kiernan, 2007, Spitz, EP 2 256 103 A1), based substrates indicate substrates that have been used to produce, at least in part due to enzyme activity, “indoxyl” (which may include substituted indoxyls—e.g.—6-cl-indoxyl, N-alkyl indoxyl, N-aryl indoxyl). They may be substituted at various carbons or nitrogen. 3-indoxyl-B-D-glucoside, N-methyl-3-indolyl-B-D-galactoside and bis-(5-br-4-cl-3-indoxy)-pyrophosphate are substrate examples reported to have been used to do this. It is thought the indoxyl nitrogen may be replaced by a heteroatom such as oxygen, sulfur, or others. The atom at this position may or may not have an alkyl, aryl, or other substituent and the carbon at the “three position” may be attached to an oxygen or a heteroatom such as sulfur, or others which may be part of or form a bridge to an enzyme recognized group.

The use of aspects of the present invention can be well suited to replace the prior use of some enzyme substrates in enzyme assays, including above mentioned examples.

### SUMMARY OF THE INVENTION

This invention provides, at least in part, novel use of some indicator enzyme substrates to detect enzyme reaction for oncology, disease, tissue, enzyme and microbe, and other biological item research and commerce related to the biological areas.

One embodiment makes use of a device, e.g., pad or gel, as a base onto which at one side a diagnostic material of at least one of a fluorogenic or chromogenic enzyme substrate is placed. A sample which may contain a specific target item, such as an organism, is applied to the diagnostic material which can be used to produce a color quality indicating the presence of a target item. UV and visible light may be used to view one or both sides of the pad or gel to detect any fluorescent and chromogenic qualities representing the target item/items. In certain embodiments, it may be unnecessary to use a pad with the gelling agent.

In another embodiment, the diagnostic material is an enzyme substrate that can be used to produce both a fluorescent e.g., fast or slow fluorescent, and chromogenic quality at one side of the base, and the quality is used to detect target items, e.g., enzyme activity.

It is an object of this invention to provide a method of detecting specific items/enzyme activity/organisms in rapid fashion.

In general, an object of this invention is to provide a method of microbial testing which is economical and provides accurate results.

Other objects will become readily apparent to those skilled in the art as a result of the following additional description.

### BRIEF DESCRIPTION OF THE DRAWINGS

The patent or application file contains at least one drawing/photograph executed in color. Copies of this patent or published patent application with color drawing(s)/photograph(s) will be provided by the Office upon request and payment of the necessary fee.

FIG. 1 is diagrammatic view of a test container used in this invention;

Photo 1 shows colonies growing on the upper surface of a pad;

Photo 2 shows the colonies in Photo 1 as detected in UV light;

Photo 3 shows the colonies in Photo 1 as detected from the bottom of a pad;

Photo 4 shows six different bacteria species on a filter;

Photo 5 shows the bacteria in Photo 4 as detected under a UV light;

Photo 6 shows the bacteria in Photo 4 as detected from the bottom of a pad;

Photo 7 shows the bacteria in Photo 4 detected due to use of a fluorogenic enzyme substrate;

Photo 8 shows the bacteria in Photo 7 as detected in UV light; and

Photo 9 shows the bacteria in Photo 7 as detected from the bottom of the filter.

### DETAILED DESCRIPTION OF PREFERRED EMBODIMENTS OF THE INVENTION

It has proven to often be beneficial in certain biological item testing (e.g.—bacteria, fungi/yeast, virus, enzyme activity, gene I.D.) to use a fluorogenic enzyme substrate or chromogenic enzyme substrate that can be used to produce a fast color quality with a solid assay. This may be done with a petri dish or other container and a pad (eg—a flat matrix that can receive and provide aqueous material) in contact against the bottom of the dish or a solid (e.g,—gelled) layer and no pad.

The pad, which may be part of the test unit, (and may be part of the Kit to be offered) may in part function to provide components to stimulate growth of target organisms. These components may comprise inhibitors (e.g.—surfactants, antibiotics) to prevent growth of non-target organisms.

The Growth Medium may be contacted to the pad which then can be used to support and/or provide the medium in dried or liquid form, dependent upon the instructions provided with each kit.

The pad may be of different thicknesses. This allows different amounts of samples to be used, dependent upon the preference of the user.

The pad may be constructed of various materials.

The thickness, as well as the composition, of the pad or gel can affect the speed of detection. A thinner pad/gel may minimize the time of detection of any fluorogenic or chromogenic quality, from the pad underside.

Thinner pads may also be less expensive than regular-sized pads. It is thought that the type of pad used (except for toxicity concerns) has not generally been an issue in the prior literature. A standard thick-type pad is commonly used for membrane filtration. It is thought that pads are not commonly used in agar and gel tests currently.

The observation for detection of any fluorescent or chromogenic qualities from the underside of the pad or gel is often particularly important when a fluorogenic or chromogenic enzyme substrate is used to produce a fast chromogenic or fluorogenic quality in a solid test. As an aspect of this invention other types of substrates are suited for use with this technique and/or other techniques. As an aspect of the original disclosure of this invention, it was found that different enzyme substrates can be used to provide new and different useful detections. For example, it is thought that traditionally, fluorogenic and chromogenic enzyme substrates have been used to produce essentially a single useful detectable quality, (e.g. a fluorescent quality or a chromogenic quality respectively) though the quality may be detected by more than one way (e.g.—naked eye or spectrophotometry). An enzyme substrate that can be used to produce more than one detectable (e.g.—color) different quality, e.g., a fluorogenic (fluorescent) quality and a chromogenic (color) quality, is herein termed a dual enzyme substrate. Use of a dual enzyme substrate to produce more than one type of detectable quality is herein disclosed and characterized as a Dual Enzyme Substrate Method. The item may be anything detectible by enzyme activity (e.g.—an aggregation of dead, non-viable, or living viable microorganisms). A dual enzyme substrate method is considered unique and is an aspect of the original disclosure of the invention. Examples of substrates that can be used in this method are 6-chloro-3-indoxyl-beta-D-galactoside and N-methyl-3-indoxyl-B-D-galactoside. In what is considered the first report of another aspect of the original disclosure of this invention, it was unexpectedly found that some indoxyl based enzyme substrates can be used to produce a slow fluorescent quality that can be used to at least to detect enzyme activity. This is here-in termed an “NF” Method. It is thought this use can be involved in production of indoxyl type compounds and/or indigo dye type compounds. (personal communication with Dr. W. Ferguson) Substrates of this type are here referred to as a “New Fluorescent” or “NF” Substrate. Examples of “NF” substrates are 6-chloro-3-indoxyl-beta-D-galactoside, and N-methyl-3-indoxyl-B-D-galactoside. Others including 3-indoxyl-α-glucoside and 5-Br-4-Cl-N-Methyl-α-glucoside are thought to be “NF” substrates and duogens too. Rambach (U.S. Pat. No. 5,846,761) reports that some indolyl substrates are used to produce “colored or fluorescent derivatives,” although the teachings of that reference are to a degree suspect because, for example, of contrary findings by others. It appears that he is using the terms “colored and fluorescent” as synonyms.

For this invention, it is thought that many of the detections can be achieved with various instrumentation (e.g. with cameras, colony counters or fluorometers), or the unaided eye.

The use of compounds, due to non-enzymatic reaction, has been reported to allow for production of a new slow diffusing fluorescent quality that can be used to detect the non-enzymatic reaction, and is expected to work as an aspect of this invention. The use of compounds attached to a silyl group (e.g.—an indolyl based-silylate) to allow for production of a slow diffusing fluorescent quality, due to reaction with fluoride ions, is an example.

This invention relates in part to the detection of items through the use of fluorogenic or chromogenic enzyme substrates.

There are many types of assays for which aspects of this invention can be used. For example, to take advantage of speed and economics, if one is testing for Escherichia coli, an easy test might involve the use of any size petri dish or other container, said container containing a gelled medium, or part of a matrix that contributes to a gel, or supporting a pad covering the bottom or a gelled matrix. A medium with a fluorogenic enzyme substrate, for example, either 4-methylumbelliferyl-beta-glucuronide MUgluc) or 4-methylumbelliferyl-beta-D-galactoside (MUgal) or MUgluc with or without various additional nutrients, (e.g. casein or soy extract) may be added to a pad. If a test sample is subjected to membrane filtration, after filtration the filter is placed onto the pad. The petri dish is incubated and monitored for detection of E. coli (target organism) by detection of spots exhibiting fluorescence, at least in part due to enzyme activity, with UV light through the bottom of the petri dish. Other target organisms and enzyme substrates that can be used to detect them by changing the target organism medium and enzyme substrates of the above procedure may comprise: for direct detection of Staphylococcus aureus, add D-Phe-Pro-Arg-β-napthylamide, which may be added to the pad and media through a micropore syringe filter or another enzyme substrate that may be used to detect this target organism is tosyl-Bly-Pro-Arg-P-nitro anilide (“Chromozyme-TH”) which may be added similarly; for detection of group A Streptococcus and Enterococcus, add any of the enzyme substrates from a group comprised of a β-naphthylamide conjugate of pyrolidony (used for the PYR test) and an amido-methyl-coumarin conjugate of pyroglutamic acid which may be added to the pad and media through a micropore syringe filter. These demonstrate an aspect of the invention which is the use of amino-peptidase and/or peptidase indicator substrates with methods of the invention (eg.—dual substrate and NF). (Bascomb and Manafi)

The following is information to accompany a series of photos used to demonstrate principles of the original disclosure of the invention.

Photo 1 shows, at 18 hours of aerobic incubation, E. coli detected by chromogenic blue and non-E. coli microbes, chromogenic pink, on the top surface of a micropore membrane filter resting on a pad with the liquid Coliscan® MF Plus medium which contains MUG gluc.

Photo 2 shows the same plate and same view as shown in Photo 1, but as seen under direct long wave UV light. The colonies match in position number on the plate. Note that virtually all the detected colonies are detected by fluorescence and that some E. coli colonies are detected by fluorescent “diffusion” representing the fast fluorescent quality representing the enzyme reaction with the MUgluc.

Photo 3 is the same plate as seen in Photos 2 and 3, but the plate is turned over and viewed from the bottom in UV so the fluorescent spots used to detect the E. coli are apparent as nearly a mirror image of the pattern of the first two photos. Note slow fluorescent quality that can be used to detect the non-E. coli is less intense from this side.

Photo 4 shows at eight hours of aerobic incubation, six different species of bacteria spotted on a micropore membrane filter on a pad with medium commercially available and sold under the name of Coliscan® MF which does not contain a significant amount of MUG gluc and manufactured by Micrology Laboratories of Goshen, Ind., The organisms illustrated are: A) E. coli (3 o'clock); B) Citrobacter freundii (6 o'clock); C) Klebsiella pneumoniae (7 o'clock); D) Enterobacter aerogenes (9 o'clock); E) Salmonella typhimurium (11 o'clock); and F) Aeromonas hydrophila (2 o'clock).

Photo 5 is the same plate and same view as shown in Photo 4, but as monitored in UV light. Note that A, B, C, D and F all exhibit slow whitish-blue fluorescence. For A, B, C and D it is evident that the slow fluoresence represents enzyme reaction with 6-cl-3-indolyl-β-D galactoside.

Stated in a different way and in this context, the use of 6-cl-3-indoxyl-β-D galactoside unexpectedly provided, at least in part due to enzyme reaction, a slow fluorescence quality which can be used to detect the enzyme reaction. It is thought that this is the first report that an indoxyl or substituted indoxyl based “NF” type enzyme substrate can be used to produce a slow fluorescent quality used to at least detect presence of a target item, (e.g.—microbe and enzyme activity). This is an aspect of the original disclosure of the invention. Some other NF substrates that can be used with this method were either based on 6-fl-3-indoxyl, 4-cl-3-indoxyl, 3-indoxyl, 5-cl-3-indoxyl, 5-br-4cl-3-indoxyl, 5-br-cl-N-methyl-3-indoxyl or N-methyl-3-indoxyl. It is thought this is the first report of use of an enzyme substrate to produce a slow fluorescent quality with a membrane filter method and is an aspect of the original disclosure. Another enzyme type that can be used this way is a quinazolinone type substrate, e.g., ELFphos. Quinazolinone type substrates have been used to produce a slow fluorescent quality, but just reported with a streak plate method for bacteria. (van Ommen Klocke, 1999). It is thought that “aldol” type substrates reported by Wick can be used before an “aldol” reaction occurs. Both the “aldol” and ELF substrates may need to be aseptically added to avoid heat damage. It is thought the original disclosure is the first disclosure to report using a NF method with a traditional microbiological membrane filtration method. Prior to the report of this application, Haugland (U.S. Pat. No. 5,443,986) used a substrate that is used to produce a slow fluorescence to detect an enzyme. It is just reported that the enzyme was detected in an aqueous non-solid test. Haughland teaches that microbes can be detected but not how to detect them. Spitz and Wick refer to an “active signalogen” causing transient green fluorescence, but infer it is unstable, and they don't appear to report using it diagnostically (EP 2,256103 A1).

Photo 6 is the same plate in Photos 4 and 5, but the plate is turned over and viewed from the bottom.

Photo 7 shows at eight hours of aerobic incubation the same six species, in the same positions on the upper surface of a filter, of microbes as shown in Photo 4, but in this case the pad is soaked with a medium, ECA Check® MF Plus, which in addition contains the fluorogenic enzyme substrate MU glue and is manufactured by Micrology Laboratories of Goshen, Ind.

Photo 8 is the same plate and same view as shown in Photo 7, but as monitored under direct long wave UV light. Note that A, B, C, D and F all exhibit fluorescence in this top view. According to prior art, for many organisms including these species, many culture tests including an agar pour plate or a membrane filter test, would not be monitored this early, but as an aspect of this invention, when detecting these species at this time, it almost certainly would have been assumed that this fluorogenic quality is produced by enzyme reaction with MUG glue. Photo 9 shows evidence that this is wrong and that enzyme reaction with 6-cl-3-indolyl-B-galactoside provided this slow fluorogenic quality which provided unexpected detection of A, B, C, D, and F, as an aspect of the original disclosure of the invention. Monitoring solid tests for early detection using substrates providing for fast or slow qualities is an aspect of this invention.

Photo 9 is the same plate as 7 and 8, but the plate is turned over and monitored from the bottom for detection of fast fluorescence quality provided by enzyme reaction with MUgluc. In this case, only A (E. coli) is detected by fast fluorescence. None of the other microbes exhibited significant fluorescence to the eye from this side of the plate because they are glucuronidase negative.

The above examples demonstrate how combinations of enzyme substrates may be used to detect microbe genera by enzyme reaction detection. As an aspect of this invention; a fluorogenic enzyme substrate can be chosen from a group comprised of substrates which can be used to produce derivatives of coumarin such as methylumbelliferone or trifloromethylumbellferone, or some indolyl based, fluoroscein, or resorufin or other fluorogenic based molecules (but not limited to these) and fluorogenic tetrazolium salts. A chromogenic enzyme substrate can be chosen from a group comprised of substrates which can be used to produce indoxyl based, nitrophenol, flouroscein, resorufin, chlorophenol red, or other chromogen based molecules and tetrazolium salts. It is thought that these substrates may be used to detect enzyme(s) chosen from a group comprising glycosidases, phosphatases, sulfatases, DNAases, RNAases, phosphoglycosidases, arylphosphatases, arylsulfatases, lipases, ATPases, apyrases and some other enzymes that can be chromogenically or fluorogenically detected, so that any combination of items might be detected such as any genus or genera of organism or cell, (e.g. bacteria, fungi or viruses).

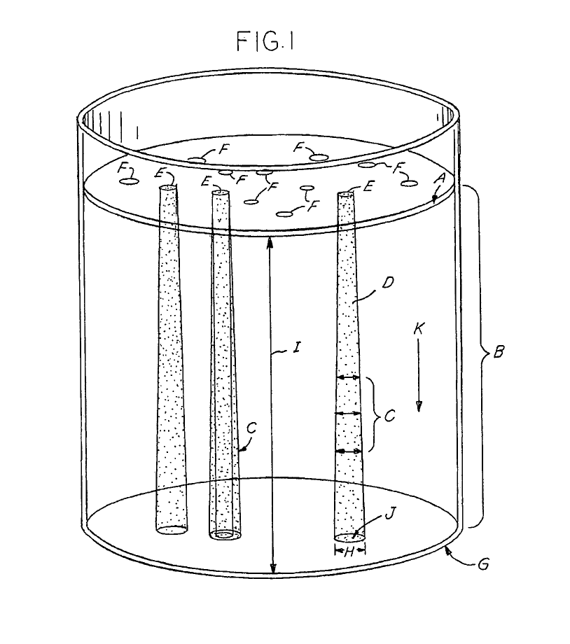

Drawing FIG. 1 shows an upright model of an apparatus that can be used in a method of the invention, not necessarily drawn to scale. It depicts an open container with organisms on a surface. “A” represents a membrane filter, if present. “B” represents a device, e.g., pad or gel or both, if present. A and B may constitute the base. C represents any lateral movement of fast quality. D represents fast quality. E represents a microbial colony, detected by fast fluorescence under UV light. It may be detected in part by the fast or slow chromogenic quality. F represents organisms that are not detected by a fast fluorogenic quality and/or fast chromogenic quality Both E and F may be detected by slow chromophoric and/or slow fluorescent quality or be other-wise detectable on the surface at the top. The bottom of the container is G. H is the width of the fluorescent spot detected through the bottom of the container, and may be represented in the drawing as wider than actual for purposes of this model. I is the thickness of the pad or gel or both. J is a fluorescent spot as detected through the bottom of the container, when it is detectable at any point during movement through the pad or gel or both. K is force moving the fluorophoric/chromophoric quality downward.

As mentioned earlier, this invention relates particularly to the detection of entities through the use of a newly defined group of enzyme substrates that we will reference as dual enzyme substrates. It is thought that no enzyme substrate has been previously described or generally used for above described dual qualities, both of which are proven useful in applications as reported in the previous descriptions of methods the present invention.

The 6-chloro-3-indoxyl-β-D-galactoside and other substrates are used for illustrative purposes concerning applications and novel uses in this discussion, but they are not meant to limit the substrates that can be used or the scope of the invention.

Prior to this invention, it is thought that no previous note has been taken of any fluorescent quality, much less one that is slow, due at least in part to enzyme reaction of 6-chloro-3-indoxyl-β-D-galactoside.

Materials were obtained from different sources: Coliscan Easygel, Coliscan Plus Easygel, Coliscan MF, Coliscan MF Plus, Pretreated Easygel dishes, 50 mm petri dishes, pads, 45 micron membrane filters, droppers, swabs, inoculation loop from Micrology Labs, 6-cl-3-indoxyl-B-glucoside, 6-cl-3-indoxyl-B-galactoside, 6-cl-3-indoxyl-Phos, 5-br-4-cl-3-indoxyl-B-glucuronide, 5-br-4-cl-3-indoxyl-B-galactoside, IPTG from Inalco, IPTglucuronide from Biosynth, IPTglucoside from Sigma, N-methyl-3-indoxyl-B-galactoside from Biosynth, N-methyl-3-indoxyl-B-glucoside from Inalco, 5 mM in water ELF 97 PHOSPHATASE SUB from Invitrogen, peptone, casein from Global Bioingredients, potassium phosphate from Amresco, beef extract from Marcor, yeast extract from BioSpringer, agar from AEP. NaCl, pyruvate and other ingredients can be obtained from Sigma. Aerobic Petrifilm™ from 3m or 3m distributor, anaerobic bags and sachets from Remel.

Basal Medium—Following is a general formulation based upon common ingredients that can be used in methods of this invention. This specific formulation should not be interpreted to be the only usable combination. In fact, there may be many variations and additions and subtractions of ingredients, depending upon the specific targets toward which the medium is aimed. There may be, in some applications of the present invention, a resuscitation/repair/pre-incubation step which may be composed of some of these ingredients.

Media ingredients (e.g. nutrients) and Sample Entities (e.g. Target organisms)

a. peptone—3-6 grams

b. yeast extract—1-3 grams

c. dipotassium phosphate—1-3 grams

d. sodium chloride—3-5 grams

e. inducer—50-200 mg. (e.g. IPTG) (optional)

f. resuscitation, repair or growth stimulating agent—0.5-2 gm (e.g. sodium pyruvate, sorbitol, etc.,) (optional)

g. buffer (optional for controlling pH)

h. selective inhibitors (optional—may include but not be limited to antibiotics, bile salts, other surfactants or related compounds). If one or more inhibitors are included, the type and amounts will vary with the desired effect upon the target organisms.

i. deionized or distilled water—1 Liter

j. Gelling or solidifying agent (optional—may include agar, pectin, alginate, or other gums) The types and amounts are determined by the desired effects.

k Amino acids (e.g.—Tryptophan—which may provide for the indole test)(optional)

l. glucose (optional)

m. pH indicator such as neutral red or crystal violet (optional)

n. kaeolin as an opaquing agent (optional)

Insisting that there can be no variation in ingredients with exact amounts of each ingredient is contrary to the art. Therefore, it should not be assumed that variations in the media formulations cited in this invention constitute a new invention.

The sample entities in the test samples may comprise various types and species of enzymes and gram positive and negative microbes, and fungi. In the following examples where the entities are bacteria, and the bacterial species used may comprise combinations of Escherichia coli, Enterobacter aerogenes, and Pseudomonas sp. E. coli and Enterobacter aerogenes are members of the Coliform Group. By definition, Coliform bacteria produce the enzyme B-galactosidase and therefore any coliform bacterial species will generally respond similarly to the Enterobacter aerogenes that was used in some of these described examples. In addition, most E. coli strains or varieties produce the enzyme B-glucuronidase (in addition to B-galactosidase) and so will generally demonstrate a unique enzyme profile among coliforms. (Where the entities are not bacteria, they may comprise other entities (e.g. enzymes, genes and fungi).

One of the embodiments of the invention is improved speed of item detection, sometimes using indicator enzyme substrates. As an aspect of this invention, evidence has been found that the generation times for target items may vary and may be affected by the presence of various factors present in the environment (e.g—media ingredients, pads, filters). Ideally the method of the invention should comprise monitoring at different times from the beginning of incubation to achieve the fastest detection, and therefore when a time such as 8-14 hours is suggested herein, this does not mean that results might not be detected sooner.

### Example 1

### An NF and/or Dual Method

a medium was made comprising the following ratios of ingredients and not necessarily 1 L was made:

a. peptone—5 grams

b. yeast extract—3 grams

c. dipotassium phosphate—3 grams

d. sodium chloride—5 grams

e. 6-chloro-3-indoxyl-beta-D-glucuronide—100 mg

f. IPTG—150 mg

g. deionized water—1 Liter

The steps of use being:

a. the above Medium was autoclaved and (approximately 1.5-2.0 mL) was added to a pad in a petri dish;

b. An aqueous sample containing Escherichia coli and Enterobacter aerogenes, (coliform bacterial species) was subjected to micropore membrane filtration. The filter was then put (top side up) on the surface of the pad;

c. The inoculated dish was then incubated aerobically at 35° C.;

d. At 8-14 hrs. incubation the dish was opened and examined under long wave UV. Colony forming units of E. coli were detected by whitish-blue fluorescence. This was an acceptably accurate count for the sample. The proof of utility of an NF enzyme substrate, e.g., 6-chloro-3-indoxyl-beta-D-glucuronide, that can be used to produce a slow fluorescent quality, which is used to at least detect target items as presence absence or quantitative, was provided by the original disclosure of the present invention.

At 22-26 hrs. there was diminished detectable fluorescence, but under ambient light, the E. coli colonies (which were detected as fluorescent spots earlier) were detectable as chromogenic red/pink dots and the Enterobacter aerogenes colonies were generally only detectable as opaque dots.

### Example 2

### A NF and/or Dual Method

The steps of use being:

a medium was made comprising the following ratios of ingredients and not necessarily 1 L was made:

a. peptone—5 grams

b. yeast extract—3 grams

c. dipotassium phosphate—3 grams

d. sodium chloride—5 grams

e. 6-chloro-3-indoxyl-beta-D-glucuronide (80 mg)+5-bromo-4-chloro-3-indoxyl-beta-D-galactoside (80 mg);

f. IPTG—150 mg

g. deionized water—1 Liter

a. The above medium was autoclaved and (approximately 1.5-2 mL) was added to a pad in a small petri dish;

b. An aqueous sample containing entities (in this case Escherichia coli and Enterobacter aerogenes bacteria) was subjected to membrane filtration. The micropore filter was then transferred (top side up) to rest on the surface of the pad; the dish top replaced and incubated aerobically at 35° C.;

d. At 8-14 hrs. under long wave UV colony forming units of E. coli were detected by a whitish-blue fluorescence, and the colony forming units of Enterobacter aerogenes did not appear fluorescent to the unaided eye. Again, the report and proof of utility of an NF enzyme substrate, (e.g., 6-chloro-3-indoxyl-beta-D-glucuronide), that can be used to produce a slow fluorescent quality, which can be used to at least detect target items, was provided by the original disclosure present invention.

e. At 22-26 hours Escherichia coli fluorescence had dissipated, but under ambient light, the E. coli colonies were now chromogenic (e.g., dark blue/purple dots) The other (Enterobacter aerogenes) coliform colonies were detected as teal chromogenic quality dots. Stated in a different way and in this context, (reference photos previously submitted with original application—Photos #1-9) the use of an enzyme substrate to produce a color quality, dissipation of the said color quality, and production of a new and different color quality is now referred to as a bulls-eye process composed of usable appearing and changing bulls-eye stages that can be used individually or in combination to detect enzyme reactions and is an aspect of the invention. It should be noted that as used herein, the term and forms of the term “diminish” or “dissipating” refers to fading of the detectable result, but not necessarily fading to the point of total extinction.

A first bulls-eye fluorescent quality stage mainly alone may indicate that the reaction has recently or just started. Any second stage that may be used for detection, chromogenic quality concomitant with fluorescent quality, may indicate that the reaction started some time ago and is most likely still occurring. (see Set II photos d & e) Any third stage that may be used for detection, chromogenic quality mainly alone, may indicate that the reaction has occurred.

Applications of the “bulls-eye” method are virtually unlimited. (See Set II photo b, Set III photo b) detection of a “bulls-eye” stage may allow detection and differentiation of different strains or species or genus of tissues/cells or microbes or stressed or injured cells or different strains of the same species or genus, each of which tends to have their own characteristic enzyme activity/growth/reproduction rate, with solid tests. Any debris present in a sample such as but not limited to meat or cheese that may provide enzyme activity may show up as an irregular shape which may differentiate it from a more circular bulls-eye microbial colony(s). This can facilitate the early identification and quantification of the different target items from non-target materials. (see Set II photo b).

### Example 3

### A NF and/or Dual Method

a medium was made comprising the following ratios of ingredients and not necessarily 1 L was made:

a. peptone—5 grams

b. yeast extract—3 grams

c. dipotassium phosphate—3 grams

d. sodium chloride—5 grams

e. 5-bromo-4-chloro-3-indoxyl-beta-D-glucuronide (80 mg)+6-chloro-3-indoxyl-β-D-galactoside (180 mg)+MU Glucuronide (75 mg)

f. IPTG—150 mg

g. deionized water—1 Liter

The steps of use being:

a. The above medium was autoclaved and (approximately 1.5-2 mL) was added to a pad covering the bottom of a small petri dish;

b. An aqueous sample containing entities (in this case E. coli and Enterobacter aerogenes coliform bacteria) was subjected to membrane filtration. The micropore filter was put (top side up) on the surface of the pad;

c. This inoculated dish was incubated aerobically for 8-14 hrs at 35° C.;

d. The dish was inverted, and it was placed under a UV light. E. coli were detected by whitish-blue fluorescent quality spots. These spots were detected by a fast fluorescent quality evidently produced by enzyme reaction with the MUgluc;

e. The dish was turned right side up, the lid was removed and it was placed under the UV light. Fluorescent whitish-blue quality spots represented the total coliform population (including the E. coli). It is thought this fluorescence was a combination of the slow fluorescent quality produced by enzyme reaction with 6-chloro-3-indolyl-β-D-galactoside (indicating coliforms, including the E. coli) and a fast fluorescent quality produced by a different enzyme reaction with MUgluc (indicating the E. coli). E. coli colonies were detectable as very tiny, pale bluish chromogenic quality dots, and most other coliforms were not yet significantly chromogenically detectable to the naked eye;

f. At 22-26 hrs. the dish was reexamined. The fluorescent quality spots detected from the underside of the dish (representing E. coli colonies) were detected as larger.

g. Upon turning the dish right side up and examining under UV, the previous fluorescence used to detect non-E. coli coliform colonies had diminished and the E. coli was still detected by some fluorescence and it had spread significantly. In ambient light, the non-E. coli coliforms were detectable by chromogenic red/pink quality dots, while the E. coli colonies were detected by chromogenic blue/purple quality; This is thought to be the first report of an example of using two different substrates to produce different fluorescent qualities, and using the qualities to detect an item(s), e.g., different enzymes, and is an aspect of the invention covered under the original disclosure. The fluorescent qualities produced at least in part by different enzymes reactions with these substrates are thought to exhibit different and distinguishable wave length ranges, and may be detected by the naked eye and instrumentation more sensitive than the naked eye.

Following is a further example with Candida yeast being an organism that is detected using a NF and/or dual method.

### Example 4

A nutrient medium was made comprising the following ratios of ingredients and not necessarily 1 L was made:

a. peptone—5 grams

b. yeast extract—2 grams

c. 6-chloro-3-indoxyl-beta-D-glucoside (80 mg)

d. deionized water—1 Liter

The steps of use being:

a. The above medium was autoclaved and (approximately 1.5-2 mL) was added to a pad a small petri dish;

b. Candida albicans yeast was spotted on a micropore filter

c. This inoculated dish was then incubated aerobically at 35° C.;

d. At 8 hrs. colony forming units of Candida were detected by whitish-blue fluorescence. Again, the report and proof of utility of a single enzyme substrate that can be used to produce an NF fluorescent quality used to detect target items was provided by the original disclosure of the invention.

f. By 19 hours, Candida colonies were detected as chromogenic pink/red appearance.

### Example 5

### A NF and/or Dual Method

Following is a further example with Enterococcus being an organism that is detected using a NF and/or dual method.

a nutrient medium was made comprising the following ratios of ingredients and not necessarily 1 L was made:

a. peptone—5 grams

b. yeast extract—3 grams

c. dipotassium phosphate—1 gram

d. sodium chloride—3 grams

e. 6-chloro-3-indoxyl-beta-D-glucoside (80 mg)

g. deionized water—1 Liter

The steps of use being:

a. The above medium was autoclaved and (approximately 2 mL) was added to a pad in a small petri dish and a micropore membrane filter was put on the pad.

b. An aqueous sample Enterococcus bacteria was spotted on the micropore filter

c. This inoculated dish was then incubated aerobically at 35° C.;

d. At 8-14 hrs. colony forming units of Enterococcus were detected by whitish-blue fluorescence. Again, the report and proof of utility of an NF enzyme substrate that can be used to produce a slow fluorescent quality used to detect target items was provided by the original disclosure of the invention.

f. At 22 hours, Enterococcus colonies were detected as chromogenic pink/red dots.

### Example 6

### A NF and/or Dual Method, Made Following Base Formula and not Necessarily 1 L, was Made (Amounts Given for 1 Liter of Base Broth)

| | |
|---|---|
| PP #3 | 10 g |
| Yeast extract | 3 g |
| K2HPO4 | 3 g |
| KH2PO4 | 1 g |
| NaCl | 2 g |
| Na pyruvate | 2 g |
| Bile salts#3 | 1.5 g |

Deionized water 1 liter

Added to the base formula were:

Formula #1: 6-Cl-3-indoxyl-α-D-galactopyranoside (conc. Of 200 mg/l)

N-Methyl-3-indoxyl-β-D-galactoside (conc. Of about 350-300 mg/l)

Formula #2: As #1, but now there is 5-br-4-cl-3-indoxyl-B-D-galactoside added (conc of 150 mg/l)

An inoculum of Salmonella typhimurium, E. coli, and Enterobacter aerogenes was subjected to membrane filtration.

The pads in 47 mm Millipore dishes were contacted with 1.5-2-0 mL of one of the two formulations and a micropore membrane filter containing the target organisms was placed on the pad surface and then incubated at 35 C, with photos taken at intervals to demonstrate the results.

At 12 hrs., the Salmonella was detected as fluorescent whitish-blue dots in 366 nm UV, the E. coli and Enterobacter as green fluorescent dots in 366 in UV.

By 18 hrs., both well differentiated fluorescent CFUs of each type were detected and (in ambient visible light) the Salmonella was detected as pink colonies, the E. coli as green colonies and the Enterobacter as lighter green colonies in Formula #1. In formula #2, the E. coli was detected as dark blue colonies in ambient with the Enterobacter as green colonies and the Salmonella as pink colonies.

### Example 7

Upon further examination, following examples have been clarified to remove misinterpretation and repetition.

Following examples show how various aspects of the invention can be applied to aerobic and anaerobic items.

a medium was made comprising ingredients in the following approximate ratios and not necessarily 1 L was made:

a. peptone—5 grams

b. yeast extract—2 grams

c. soy extract—2 grams

c. beef extract—2 grams

d. sodium chloride—3 grams

g. deionized water—1000 ml

The medium was autoclaved and various substrates were added to compose different media. In a case where ELF based substrate was used, 0.5 ml diluted substrate in water was added to 5.5 ml of medium to make that medium. In other cases around 300-500 mg/liter was added to the medium to make those media.

1. 0.5 ml of a 1:10 dilution of “5 mM in water” of “ELF 97 PHOSPHATASE SUB”
2. 6-chloro-3-indolyl phosphate
3. 6-chloro-3-indolyl-B-D-glucuronide
4. 6-chloro-3-indolyl-B-D-galactoside
5. 6-chloro-3-indolyl-B-D-glucoside
6. N-methyl-3-indolyl-B-D-galactoside

Media was made were each separately added to one (for each medium) separate quadrant cut from a pad covering the bottom of a small petri dish for each medium. A quadrant cut from a 0.45 pore size membrane filter was put on a pad quadrant, inoculated and incubated at 35° C.

Microbes used to inoculate were chosen from a group comprised of; E. coli ATCC 25922, Staph aureus, B. fragilis ATCC 25285, Enterobacter aerogenes and C. perfringens ATCC 13124.

E. coli was detected by slow sapphire/green fluorescence and Enterobacter aerogenes by green fluorescence using Medium with substrate #1 and substrate #3 in aerobic incubation. It is thought this is the 1st report of use of two different kinds of substrates to produce contrasting slow fluorescence with a solid test and is an aspect of the invention.

E. coli was detected by slow green fluorescence using Medium with substrate #1

E. coli was detected by slow dark green fluorescence using Medium with substrate #6 in aerobic incubation.

Staph aureus was detected by slow green fluorescence using Medium with substrate #1 in aerobic incubation.

E. coli was detected by slow green fluorescence using Medium with substrate #1 where in the substrate had been heated, and though usable, background fluorescence on the un-inoculated filter areas with medium was detected in aerobic incubation.
A. E. coli was detected by slow green fluorescence using Medium with substrate #1 when inoculated in an anaerobic bag with an anaerobic satchet.
B. fragilis was detected by fluorescent quality and by a chromogenic dull bluish-grey quality with virtually no hint of pink or red with each of medium with substrate #1, medium with substrate #2, medium with substrate #3 and medium with substrate #4, with the strongest fluorescence with medium with substrate #3 and the weakest with medium with substrate #4 when inoculated in an anaerobic bag with an anaerobic satchet.
C. perfringens was detected with each of medium with substrate #1 and medium with substrate #4 by fluorescent quality and chromogenic dull grey quality, with the strongest fluorescence in medium with substrate #1 when inoculated in an anaerobic bag with an anaerobic satchet.
   When indolyl based substrates were used anaerobically they seemed to provide fast diffusion of the fluorescent quality. This is another aspect of the invention involving the use of indolyl based enzyme substrates to provide at least one fast fluorescent quality that can be used to detect an enzyme reaction. It is thought that as an aspect of this invention, the assays they are used in can be monitored earlier, e.g., 8-10 hours, or before much diffusion has occurred of the detected appearance. In addition, it is thought that tetrazolium or diazonium salts or other oxidants can be used in the media which may provide slow fluorescent and chromogenic qualities as an aspect of the invention.

Upon close analysis the nature of any compound responsible for the NF slow quality is uncertain. Indoxyl based substrates might not be able to be used to produce insoluble colors or fluorophores. A side chain on the indolyl at the #6 position may not be the cause of insoluble product. Some currently known indoxyl based enzyme substrates are dual enzyme substrates and NF substrates. Both the dual and NF nature of 6-cl-3-Indolyl-B-D-galactoside, is thought to be due in part to the substituent at the #6 position on the Indolyl substrate. The functional characteristics of dual enzyme and NF substrates are not limited to this specific group of compounds. For instance, the substituted nitrogen of the N-methyl-3-indolyl-B-D-galactoside, is thought to contribute to its dual and NF nature.

Another example of this invention is the use of an enzyme substrate to produce a slow fluorescent quality, the NF method and/or the dual enzyme method with, on and in, a solid assay, (e.g.—gel medium without a necessary pad or necessary membrane). (Photos “a” and “b” Set VI).

### Example 8

a. A solution consisting of the following proportions of ingredients was made. (15 gm agar 5 gm Peptone, 3 gm yeast extract and 3 gm dipotassium phosphate and 200 mg N-methylindoxyl-B-D-galactopyranoside/liter of deionized water). And not necessarily 1 L was made.

b. The agar media were heat sterilized and a petri dish was poured. As it started gelling, it was inoculated below the surface with target organisms (Escherichia coli), and following most gelling, was inoculated on the surface with Escherichia coli and incubated aerobically at 35° C.

c. The dish was monitored for detection of Escherichia coli in UV light and in ambient light.

d. E. coli that grew in the matrix and on the surface of the matrix were detected by both slow green fluorescent and slow green chromogenic qualities. (Set VII Photos a, b).

### Example 9

a. A dish of Coliscan® Easygel® (contains enzyme substrates 5-bromo-4-chloro-3-indolyl-B-D-glucuronide and 6-chloro-3-indolyl-B-D-galactoside) was poured. Prior to most gelling, the medium was inoculated below the surface with E. coli and, following gelling, both on the gel surface and on the surface of a micropore membrane that was deposited on the gel at. It was aerobically incubated at 35° C.

b. The dish was monitored for detection of E. coli both in UV light and in ambient light.

c. E. coli that grew in the matrix and on surface and on the membrane surface were detected by slow fluorescent and slow chromogenic qualities. (Set VII Photos e, f).

An aspect of this invention is the use of an enzyme substrate to produce a slow fluorescent quality, the NF, and/or a non-NF dual substrate (which may be impregnated in or provided by a strip or piece of support, e.g., swab, or paper, for spot testing (some spot tests are toxic because of the solvent (Biolife brochure for MUAG test) or confirming purposes, etc.) to produce a fluorescent quality, or a fluorescent quality and a chromogenic quality to detect a target item (e.g.—E. coli) wherein the substrate is a main carbon source, essentially the only medium ingredient besides water.

### Example 10

a medium was made comprising the following ratios of ingredients and not necessarily 1 L was made:

a. peptone—5 grams

b. yeast extract—3 grams

c. dipotassium phosphate—3 grams

d. sodium chloride—5 grams

g. deionized water—1 Liter

The steps of use being:

a. the above Medium was autoclaved and (approximately 1.5-2.0 mL) was added to a pad in a petri dish;

b. A micropore filter was then put (top side up) on the surface of the pad and spotted with E. coli;

c. The dish was then incubated aerobically at 35° C.;

6-chloro-3-indoxyl-beta-D-galactoside was dissolved in sterile water or broth media and spotted on a colony. Fluorescence was detected within 2 minutes and pink chromogenic quality was detected in 5-10 minutes. Similar results were obtained with IBDgluc, 5br-4cl-3-indoxylglu, N-methyl-3-indoxylgal and 6-chloro-3-indoxyl-beta-D-glucuronide, 6-chloro-3-indoxyl-beta-D-galactosid, ELF-97 phosphatase sub, ONPG and MUgal on similar solid media for the appropriate fluorescences and chromogenic qualities. This shows that some enzyme substrates can be used in nontoxic solution of water or broth media as rapid spot tests that don't damage the organisms being tested as an aspect of the invention.

### Example 11

a. Devices (in this case sterile swabs) were moistened with 6-chloro-3-indolyl-B-D-galactoside in deionized water, providing about 2-3 mg 6-chloro-3-indolyl-B-D-galactoside in each swab.

b. A first device was touched directly to a colony on a solid nutrient medium, the second device inoculated with a loop-full of a nutrient broth culture of E. coli, additional devices inoculated with deionized water containing E. coli bacteria, and a control device moistened with sterile deionized water were placed in a container and aerobically incubated at 35° C.

c. At 1 hour E. coli was detected with the first device by pink chromogenic quality, and blue fluorescent quality. E. coli was detected in all devices but the control by blue fluorescence quality.

d. At 2 hours E. coli was detected by pink chromogenic quality in the first and second devices.

No significant ingredients other than enzyme substrate and water were needed.

Another embodiment of the invention is the combined use of at least two indicator enzyme substrates chosen from a group comprising NF substrates, including dual/multigen substrates that are NF substrates. At least one substrate is used for each different enzyme type, and the said substrates are chosen to each produce at least slow fluorescent quality that contrast with each other. They can be used to detect and/or differentiate one, two or more items (e.g.—enzyme reactions and microbes) with one solid assay. This is thought to be the first report of this method. Suitable substrates are thought to include, but are not limited to, 6-chloro-3-indoxyl-B-D-glucuronide, N-methyl-indolyl-B.D-galactopyranoside, and 6-fluoro-3-indoxyl-B-D-galactoside, 4-chloro-3-indoxyl-B-D-galactoside, 5-chloro-3-indoxyl-B-D-galactoside, and 3-indoxyl-B-D-galactoside substrates. The following Example 12 is an example of this embodiment.

### Example 12

An example uses a NF and a dual method (Set III photos)

The steps of use being:

A medium comprising the following ratios of ingredients was made and not necessarily 1 L was made

a. peptone—5 grams

b. yeast extract—3 grams

c. dipotassium phosphate—3 grams

d. sodium chloride—5 grams

e. 6-chloro-3-indolyl-beta-D-glucuronide (80 mg) and N-Methylindoxyl-beta-D-galactopyranoside (75 mg)

f. IPTG—150 mg

g. deionized water—1 Liter

The medium was made and autoclaved.

1.5-2 mL of the medium was added to a pad in a suitable container.

An aqueous sample containing, in this case, #1 glucuronidase positive/galactosidase positive bacteria (Escherichia coli), #2 glucuronidase positive/galactosidase negative bacteria (Salmonella sp.), and #3 glucuronidase negative/galactosidase positive bacteria (Enterobacter aerogenes) was subjected to membrane filtration. The membrane was put (top side up) on to the pad and the container was incubated aerobically at 35° C.

The assay was monitored for detection of organisms by using photographing, and the eye.

At 6-9 hrs. some #1 were detected by slow fluorescent whitish-blue quality (see Set II photo a) Few if any organisms were detected by chromogenic quality provided by use of indicator enzyme substrates.

At 10-14 hrs. most #1 & 2, differentiated to some degree by colony size, were detected by slow fluorescent blue quality and most #3 were detected by slow fluorescent green quality. Some #1 were detected by slow chromogenic dark purple quality.

At 16-18 hrs. most #1 were detected by slow fluorescent blue quality and by slow chromogenic dark (almost black) purple quality, most #2 were detected by slow whitish-blue fluorescent quality and by slow chromogenic pink quality, and most #3 were detected by slow fluorescent green quality (see Set III, photo b) and by slow chromogenic green quality. (see Set III, photo a). At this time most colonies were detectable. A very dark, smaller compact appearance of many #1 was unexpected because it is thought to be a result of the combined Pink and the light Green chromogenic appearances. It is thought that through the present invention this is the first report and proof of usefulness of combining a N-methyl-3-indolyl based substrate and a hydrogen-N-substituted (not N-methyl)-3-indolyl based substrate to produce a more compact and unexpected chromogenic slow quality with an assay. These stages can be detected by instrumentation (e.g.—photographed). (see Set III photos with interpretations).

N-methyl-3-indoxyl substrates have been used for fluorescent detection but it is thought the substrates were heat labile, not reported with a solid medium, and not reported to provide slow chromogenic quality. (Guilbault, 1965).

Another example of this embodiment used Elf-97 phos and 6-cl-B-glucuronide to detect and differentiate E. coli and Enterobacter with a solid assay.

It is known that there are microbes that can be detected by exhibiting natural chromogenic quality; (e.g.—Staphylococcus aureus detected by a yellow quality) or naturally occurring fluorescence in Ultraviolet light (e.g.—Pseudomonas aeruginosa, Pseudomonas fluorescens and Pseudomonas putida) all of which are commonly found in water and soil and which are associated with various diseases. Prior art has found that “the fluoresence signal is usually too small for visual detection” (Manafi, 1996). It is known that other types of microbes may also possess a fluorescent property.

An aspect of the invention is combined use of natural chromogenic and/or fluorescent quality provided by a biological item and a fluorogenic and/or chromogenic enzyme substrate(s) to detect and differentiate some items, (e.g.—E. coli, Coliform and some Pseudomonads). These Pseudomonads generally possess a fluorescent and chromogenic quality that can be differentiated from qualities used to detect other microbes. Sometimes a bullseye method can be used.

### Example 13

A medium with the following ingredients in the following ratios was made and not necessarily 1 L was made:

a. peptone—5 grams

b. yeast extract—3 grams

c. dipotassium phosphate—3 grams

d. sodium chloride—5 grams

e. 6-chloro-3-indolyl-beta-D-glucuronide-80 mg

f. IPTG inducer—150 mg

g. deionized water—1 Liter

The steps of use being:

a. 1.5-2 mL of the above medium was added to a pad that was in a small petri dish.

b. An aqueous sample containing entities (in this case, Escherichia coli coliform bacteria and Pseudomonas aeruginosa bacteria known as “target organisms”) was subjected to membrane filtration. The micropore membrane was then put (top side up) on the pad, and the petri dish was closed and incubated at 35° C.

c. At 6-8 hrs. until about 18-20 hrs. the E. coli were detected as slow fluorogenic whitish-blue quality by using instrumentation, (e.g., photographing), and/or the naked eye.

d. At 22 hrs. E. coli were detected by slow chromogenic pink quality and Pseudomonas by fluorescence by using instrumentation, (e.g.,—photographing), and/or the unaided eye. (see Set IV photos and interpretations).

It is thought that some tetrazolium salts (e.g. 5-cyano-2,3-ditolyl tetrazolium chloride or triphenyltetrazolium chloride) may be used alone or in combination with some enzyme substrates as an aspect of the invention to provide faster detection and differentiation of items.

An important aspect of this invention is uses for various indicator enzyme substrates. Some indolyl based, and some other indicator enzyme substrate types have been shown to withstand high temperatures (121° C.) without significant loss of effectiveness. This may not be true of all other indicator enzyme substrate types. This can be an important factor in media production for certain applications of the present invention.

It has been found that some indicator enzyme substrates can be used to allow for more than one useful detectable quality in assays. These substrates were termed “dual” enzyme substrates, and now the additional related terms: duogens, digens, multiple enzyme substrates, multigens, polygens, and so on are used herein to describe them. In the first disclosure of the invention 6-chloro-3-indolyl-beta-D-galactoside was used as a dual substrate. Two more examples found to work are Resorufin-B-galactoside and di-B-galactosyl fluoroscein, and it is thought that the invention provides the first report of use of the dual nature of these substrates in an assay.

Aspects of this invention are use of a dual substrate and use of an indolyl based substrate producing fluorescent quality in a broth medium. (Some indoxyl based substrates have been reported to provide fast fluorescent appearances in broth type non-solid tests. but it is thought the detection was not by the naked eye. In addition the test instructions were not adequate.) They may be applied as a presence/absence or MPN test. [Different substrates including 6-cl-3-indoxyl-acetyl-B-D-glucosaminide for some fungi or 6-cl-3-indoxyl-B-D-glucoside for Enterococcus and appropriate inhibitors such as antibiotics may be used but are not meant to limit the variations.]

### Example 14

Two Media formulations ratios were used to compare these aspects to use of a traditional Mugal and not necessarily 1 L was made.

a. peptone—5 grams

b. yeast extract—3 grams

c. dipotassium phosphate—3 grams

d. sodium chloride—5 grams

e. 6-chloro-3-indolyl-beta-D-galactoside 160 mg or MU galactoside 100 mg

f. IPTG inducer—150 mg

g. deionized 1 Liter

100 ml of the above medium using 6-chloro-3-indolyl-beta-D-galactoside was made and 10 mL put in a test tube.

The medium was inoculated with target organisms, E. coli, and incubated and monitored for detection.

At 8-12 hrs. E. coli was detected by fluorescence.

At 14 hrs. after shaking the test tube a chromogenic pink quality was detected.

100 mL of the above medium using MU galactoside was made and 10 mL put in a test tube.

The medium was inoculated with target organisms and incubated and monitored for detection.

At 8-12 hrs. E. coli was detected by fluorescence. (See Set I, Photos A and B of broth cultures of E. coli).

In addition, it was found with the present invention with a broth medium containing N-methyl-3-indolyl-B-D-galactoside and B-gal positive bacteria allowed enzyme reaction and a detectable green quality that dissipated and then reappeared upon agitation.

In addition, a dual method may be used with a broth format. MUGal can substitute for an indoxyl based galactoside substrate in this experiment. If bile salts #3 are added to the medium components before or after water, or any other appropriate inhibitor or antibiotic is used, the medium may not need to be aseptic before use.

An aspect of this invention is use of some substrates, with a dual method and/or a non-dual indolyl-based substrate, and/or non-sterile ingredients and use of appropriate inhibitors. (e.g.—antibiotics, bile salts, sodium lauryl sulfate) to repress non target items. Alternatively, this aspect may make use of a method well known in the art where in the test sample/medium mix may be dispensed into wells or containers to “quantify” the numbers of coliform bacteria or other items that may allow detection and estimates enumeration as the “MPN” (most probable number).

### Example 15

A basic medium was formulated as follows and not necessarily 1 L was made:

| | | |
|---|---|---|
| Proteose peptone #3 | 2 | gm |
| Yeast extract | 2 | gm |
| Dipotassium PO4 | 3 | gm |
| Monopotassium PO4 | 1 | gm |
| Sodium pyruvate | 2 | gm |
| Sodium lauryl SO4 | 100 | mg |
| Tryptophane | 2 | gm |
| IPTGal | 100 | mg |
| Deionized water | 1 | liter |

The basic medium was used to make 3 different media containing enzyme substrates:

1. 100 mL of basic medium+15 mg of X-glucuronide

2. 100 mL of basic medium+30 mg of Salmon gal (6-chloro-3-indolyl-B-D-galactoside)

3. 100 mL of basic medium+8 mg of X-gluc and 15 mg of salmon gal.

About 10 ml of each of the three different media were added to 13 mL tubes and one tube (tube 1) was inoculated with Enterobacter aerogenes, the second (tube 2) with Escherichia coli, and the third (tube 3) with both bacterial species.

Kovac's reagent detects tryptophanase reaction and indoxyl based substrate can be used to provide fluorescent quality for detection of B-D-galactosidase. This is considered part of the invention. Kovac's is added after incubation to the broth and turns the top a cherry red if positive. The tryptophanase reaction is positive for virtually all species of Escherichia coli.

The results were:

Medium #1—

Tube 1—bacterial growth, but no production of fluorescence or chromogen

Tube 2—bacterial growth with initially strong bluish fluorescence followed by teal green chromogen.

Tube 3—bacterial growth with initial fluorescence followed by teal green chromogen.

Medium #2—

Tube 1—bacterial growth with strong blue fluorescence followed by pink/red chromogen

Tube 2—bacterial growth with strong blue fluorescence followed by pink/red chromogen

Tube 3—bacterial growth with strong blue fluorescence followed by pink/red chromogen

Medium #3—

Tube 1—bacterial growth with strong blue fluorescence followed by pink/red chromogen

Tube 2—bacterial growth with strong blue fluorescence followed by blue chomogen

Tube 3—bacterial growth with strong blue fluorescence followed by blue chromogen

This medium also gives very good, reliable results with Kovacs reagent for the presence of Indole that confirms E. coli.

This approach to P/A detection of microbes can utilize known components in a unique way or may also utilize newly developed components. It has the advantage of allowing very large test samples to be utilized or it can be used in a format that will utilize very small (1 mL or less) test samples. It can be obtained as a pre-measured formulation in a supplied container or as a granulated formulation, which can be deposited into a container of choice.

Manafi teaches that “the disadvantage of incorporating MUG in agar is that fluorescence diffuses rapidly into the surrounding agar substrate and therefore the plates have to be read after overnight incubation usually. Several attempts have been made to simultaneously detect coliforms and E. coli in water, using o-nitrophenyl-B-D-galactopyranoside (ONPG) and MUG (Edberg et al., 1988, 1989, 1990). However, it was observed that also chromogenic nitrophenolic substances such as ONPG or p-nitrophenol-B-D-glucuronide (PNPG) diffuse easily through solid media. Therefore, these substrates cannot be used in solid media.” (Manafi, 1991) Bascomb and Manafi, 1991, defines overnight incubation as requiring as much as 24 h, “ . . . this adds an overnight incubation period to the duration of the test. Therefore, the so-called 15-minute test in reality may require 24 h”. (Manafi, 1991) In contrast, with the present invention it has been found that fluorogenic substrates producing a fast fluorescent quality, (often contrasting qualities) various ones (e.g.—di-fluoroscein B-D galactoside, and MUGluc) can be used to detect separate coliform CFUs with a solid test method within 12 hrs. and it is obvious that they can be used together for detection and differentiation of enzyme activity earlier than 18-24 hr by eye or with instrumentation with a solid or liquid assay.

The next three sentences are described aspects of the invention. ONPG can be used to detect and quantify coliforms in a solid assay.

N-methyl-B-galactoside and MUgluc can be used together to detect E. coli and Enterobacter by 19 hrs.

pH indicators used to detect sugar fermentation are expected to work with the invention.

### Example 16

6-chloro-3-indolyl-B-glucuronide and E. coli was added in 1 ml water to an aerobic Petrifilm™ brand device from 3m corporation. E. coli was detected using a dual method and a NF method in with the aerobic Petrifilm™ brand device of the 3m corporation and it is thought that these methods, and early detection using any indicator substrate, with or without a photomultiplier or different magnification system can be used with various Petrifilm™ devices and these devices can be made with dual and/or “NF” substrates, showing the versatility of an aspect of the invention. The above was repeated substituting ELF-97 phosphatase sub for the 6-chloro substrate and after incubation E. coli was detected by slow green fluorescence.

The same or different substrates may be used, e.g., fluorescyl-di-B-D-galactoside together and/or Mugluc or Mugal and Mugluc together, for each method above, differentiated by the contrast and/or comparison of intensities of one or more fluorogenic and chromogenic qualities. At early stages, observation or images taken of an assay at different times by the naked eye and/or instrumentation can be used to obtain diagnostics e.g., quantification and qualification of injured organisms, and other details.

It is known in the prior microbial assay art that current methods for detecting and/or differentiation of fungi often need a long incubation period, typically 18-72 hrs or more as growth may be much slower than the growth common to many bacterial entities. However, unpublished work for this invention with various molds using indicator enzyme substrates including NF and dual substrates, show that early detection, differentiation and enumeration may be achieved within 24 hr. (see Set V photos) It is thought that Chromogenic and Fluorogenic tetrazolium salts may sometimes also be used to rapidly detect fungi.

The use of one or more indicator enzyme substrates, e.g., NF and/or others for different qualities and/or enzymes, in a single e.g., medium, may provide the detection and/or differentiation of various types or species of molds, mold tissues, hyphae or spores, depending on their enzyme profiles The rate that different types of mold grow and varying times and amounts of different enzymes production can also be factors in the detection and differentiation of mold.

Substrates that can be used to provide results by reaction with Phosphatase, B-glucosidase and acetylglucosamidase and acetylgalactosamidase may be used as fungi detectors. This is thought to be the first described method using a fluorogenic substrate for rapid detection and identification of mold and is an embodiment of this invention. Inhibitors may be used. eg penicillin or other appropriate antibiotics or materials. An inhibitor can ideally sufficiently suppress non-target entities but not significantly suppress target fungal and any other target entities. Inducers may be used. Many combinations of mold and other microbes may be detected and differentiated in one assay with this method.

### Examples Completed for Mold

### Example 17

The steps of use being:

a. A Basal Medium was made comprising the following ratio of ingredients. Deionized water (1 liter), Peptone (5 g./L), Yeast Extract (3 g./L) and Potato Extract (3 g./L). Three different media formulations were used by the addition of a different enzyme substrates to each of one aliquot of the basal medium.

b. 6-chloro-3-indolyl-B-phosphate (180 mg/L) was added into one aliquot of the Basal Medium, MU-glucoside (100 mg/L) was added into a second aliquot of the Basal medium, and MU-galactoside (100 mg/L) was added into a third aliquot of the basal medium.

c. About 1.5-2 ml of each medium was added to each separate pad in separate petri dishes, and a micropore membrane was placed on each pad. (The above media with a gelling agent may work also, e.g.—on or in agar or an Easygel® test.

d. Mold; Aspergillus niger and Stachybotrys, (e.g.—spores and/or hyphal fragments) were then contacted with each micropore membrane and the containers was closed and incubated at 25-30° C.

In 24-36 hrs. or less Aspergillus niger and Stachybotrys Mold was detected by fluorescent quality in all three and MU-glucoside media, respectively (see SetV photos) by instrument use (e.g.—photography), and some by the unaided eye.

For this invention it is thought target item detection may be enhanced by promoting access of substrate into a target entity and/or to a target enzyme. In a microbial assays, agents may be used that permeate or induce permeability to the membrane of bacteria Vaara teaches certain previously known materials may work, including cationic agents such as Polymyxin, Polymyxin nonapeptides, and others, though Polymyxin nonapeptides aren't usually lethal.

Luminescent enzyme substrates and fluorogenic substrates producing fast qualities have been used to shorten detection time to 6.5 hours, but often requires three steps and x-ray usage, and may destroy the detected target item. (Van Poucke, Nelis 1995). One bacterium type was targeted with one indicator enzyme substrate, with or without extra fluorescent dye. Van Poucke teaches that medium ingredients, such as yeast extracts, peptones, and others, can cause background fluorescence, making it harder to detect the substrate/enzyme reaction produced fluorescence. It has been recognized as an aspect of this invention that this can be true for chromogenic substrates too. Van Poucke (1997) concludes “This study presents evidence for the unfeasibility of enzymatic presence-absence tests to detect one total coliform or one Escherichia coli organism in 100 ml of drinking water within a working day. The results of field trials with prototype chemiluminometric procedures indicated that the sensitivity-boosting measures that are essential to achieve the required speed compromise the specificity of the tests.” Berg teaches that coliforms may be detected in around 6-8 hours using a membrane filter system, (Berg, 1988, U.S. Pat. No. 5,292,644, U.S. Pat. No. 5,518,894), but 41.5° C. incubation may be needed, and the results may not be an accurate count at this time. VanPouke's test is too sensitive and may give false positive results, and Berg's test may give false negative results. We have found that in part by combining any added inducer with any combination of substrates used to produce slow or fast qualities, detection can be accurate and achieved early, especially if certain inhibitors are used to prevent false positives. Premature counts that are too early may be matched to predicted later accurate counts based upon comparison studies. This may be possible for various microbe tests.

### Example 18

| | | |
|---|---|---|
| Peptone | 4 | g |
| Yeast extract | 2 | g |
| K2HPO4 | 3 | g |
| KH2PO4 | 1 | g |
| Nacl | 2 | g |
| Tryptophane | 0.7 | g |
| Sodium lauryl sulphate | 200 | mg |
| Sodium pyruvate | 2 | g |
| 6-chloro-3-indoxyl-β-D-galactoside | 100 | mg |
| IPTG | 200 | mg |
| Deionized water | 1000 | mL |

The above Medium was autoclaved and (approximately 1.5-2.0 mL) was added to a pad in a petri dish. An aqueous sample containing Escherichia coli was subjected to membrane filtration. The filter was then put (top side up) on the surface of the pad. The inoculated dish was then incubated aerobically at 35° C.

Within 8 hrs incubation the dish was opened and examined under a long wave UV. Colony forming units of E. coli were detected by whitish-blue fluorescence.

Following paragraphs address further aspects of the invention:

There are ingredients commercially available meant to enhance enzyme reaction detection which may be used with this invention. Some are referred to and reputed as inducers, or enzyme inducers and may be referred to as inducers in this disclosure. In prior art, Isopropyl-B-D-thio-galactopyranoside, (IPTG) has been used when assaying for B-galactosidase. It is thought that many ingredients may serve as an enzyme inducer, including indicator substrates (e.g.—chromogenic and fluorogenic substrates). Examples may include different isopropyl thio and methyl-O-based compounds including glycosides In addition, inducers may be used in media without chromogenic or fluorogenic substrates. Sugars such as lactose have been used as intended inducers, though the efficacy is doubted in some cases.

### Example 19

One liter of “base medium” was made as follows:

| | | |
|---|---|---|
| Proteose Peptone #3 | 4.0 | gm |
| Yeast Extract | 2.0 | gm |
| K2HPO4 | 3.0 | gm |
| KH2PO4 | 1.0 | gm |
| NaCl | 2.0 | gm |
| Na Pyruvate | 2.0 | gm |
| Na Lauryl SO4 | 200 | mg |
| Deionized Water | 1000 | mL |

Ten bottles of 100 mL of base were made and sterilized at 121 C. Unused base was frozen.

Four aliquots of base medium (50 mL each) were prepared as follows:

1. added 6-Cl-3-indoxyl-B-D-glucuronide (120 mg/L conc) plus IPTglucuronide (5 mg),

2. added 6-Cl-3-indoxyl-B-D-galactoside (200 mg/L conc) plus IPTgalactoside (5 mg),

3. added 6-Cl-3-indoxyl-B-D-galactoside and 6-Cl-3-indoxyl-B-D-glucuronide plus IPTgal &IPTgluc (same concentrations as above),

4. added 6-Cl-3-indoxyl-B-D-glucoside (200 mg/L conc) plus IPTglucoside (5 mg),

5-7 were the same as 1-3 except lacking inducer. The amounts of enzyme substrates were identical because the substrates were added to individual bottles of 100 mL of base and split to make the individual bottles of 50 mL before the inducer was added to #1-3),

8. same as #4 except without inducer.

#1, 2, 3, 4, 5, 6, 7 and 8 were each dispensed into dishes containing sterile pads (about 1.5-2.0 mL medium/pad).

1 mL of E. coli (ATTC 25922) and Enterobacter aerogenes in approx. 10 mL of diluent were filtered through a 0.45 um Millipore membrane filters and one such prepared filter was placed on the surface of each of #1, 2, 3, 5, 6 and 7.

Dishes 4 &8 were inoculated with Enterococcus faecalis.

At 8 hr. incubation at 35 C, the following was detected:

#1 4-5 faint fluorescent colonies (CFU),

#2 42 fluorescent CFU,

#3 75 fluorescent CFU,

#4 3-4 faint fluorescent CFU,

#5 0 CFU detected,

#6 0 CFU detected,

#7 20 CFU detected, and

#8 0 CFU detected.

From the above results, it appears that each of the media containing inducer (#1-4) showed earlier detection of target organism appearance than the corresponding media not containing inducer (#5-8).

By 24 hrs. incubation time, the differences among the two sets of media were not nearly as evident, although the total CFUs still favored the media with inducer.

Virtually any inhibitor(s) of non-target items may be used with this invention. Inhibitors can include antibiotics, (e.g.—cefsulodin (may harm coliforms) and penicillin), bile salts and related compounds (e.g.—sodium deoxycholate, sodium lauryl sulfate), and various surfactants (e.g. Tergitol-7).

It was found that amounts of some ingredients in an assay may be minimized to encourage some target organisms to utilize other ingredients, e.g., specific enzyme substrates.

Sugars such as but not limited to sorbitol, maltose, mannitol, melibiose and compounds such as sodium pyruvate (or pyruvic acid) may be used in any combination with methods of the invention to improve detection of some target items Temperature may be used too, as appropriate in a given application. Methods of the invention may include use of a separate resuscitation/preincubation step to improve detection of some items such as microbes.

Most probable number tests may be used to determine probable final counts for different assays with systems that can be devised to detect early count results. A reference system may be made (e.g—with a membrane filter microbial assay) to use early detected identification and counts at various incubation times and temperatures and other conditions to predict probable counts using actual predetermined probable count correlations for various samples.

The present invention may use, but does not require a photomultiplier, a viable microorganism, or an enzyme substrate that should be capable of reacting with most enzymes.

### DEFINITIONS OF TERMS

1. Assay—an object and/or action that may allow for one or more of analysis, maintenance, or growth of a specific component.

2. Solid Assay (Test)—An assay (e.g.—agar pour plate, agar streak plate, membrane filtration) performed or conducted with a self-supporting matrix, which may comprise a tissue, a gel, membrane, agar, plastic film or other support.

### REFERENCES

Berg, J D., Fiksdal, L., Rapid Detection of Total and Fecal Coliforms in Water by Enzymatic Hydrolysis of 4-Methylumbelliferone-B-D-Galactoside, Appl. Environ. Microbiol. 54:8 Aug. 1988, p. 2118-2122.
Biolife Italiana Srt. MUAG CANDI KIT
Berg U.S. Pat. No. 5,292,644
Berg U.S. Pat. No. 5,518,894
Spitz European Patent Appl: EP 2 256 103 A1
Wick PCT WO 2009/061733 A2
Wick US pub no US2010/0291591 A1 Nov. 18, 2010
Nelis U.S. Pat. No. 5,861,279
Van Poucke, S O and H J Nelis, Limitations of highly sensitive enzymatic presence-absence tests for detection of waterborne coliforms and Escherichia coli. Appl Environ Microbiol 1997, February, 63(2) p. 771-774.
Builbault, G G, N-METHYL INDOXYL ESTERS AS SUBSTRATES FOR CHOLINESTERASE Analytical Letters, 1(6), 365-479, (1968)
Kiernan, J A, Indigogenic substrates for detection and localization of enzymes. Biotechnic & Histochemistry 2007 82(2): 73-103.
Van Ommen Kloeke, F. Novel method for screening bacterial colonies for phosphatatase activity. J Microbiol Methods 38(1999) 25-31.
Van Ommen Kloeke, F. Localization and identification of populations of phosphatase-active bacterial cells associated with activated sludge flocs. Microbial Ecology (1999) 38:201-214.
Bascomb, S. and M. Manafi. Use of Enzyme Tests in Characterization and Identification of Aerobic and Facultatively Anaerobic Gram-Positive Cocci. Clinical Microbiology Reviews (1998) 11(2):318-340.

Although the present invention has been described above in detail, the same is by way of illustration and example only. Those skilled in the relevant art will now understand from this disclosure that, for example, each and every “aspect” of the present invention is not required to be used in every application of the present invention and that various modifications based upon the examples and aspects stated are contemplated by this invention. Accordingly, the spirit and scope of the present invention are limited only by the terms of the following claims.

What is claimed is:

1. A method of detecting an enzyme reaction, using an enzyme substrate diagnostic material that can be used to provide for at least two different detectable qualities, wherein at least one of these qualities is slow fluorescent if it is the only quality being monitored for detection.

2. A method of detecting an enzyme reaction according to claim 1, comprising the steps of:
Obtaining a sample,
Contacting said sample with an enzyme substrate diagnostic material that can be used to provide for at least a fluorogenic quality and a chromogenic quality that each can be used to identify said enzyme reaction,
Monitoring results of said contacting to detect at least presence or absence of fluorogenic quality, said presence t representing said enzyme reaction, and
Monitoring the result of said contacting to detect at least presence or absence of chromogenic quality said presence representing said enzyme reaction.

3. The method according to claim 2 wherein the enzyme substrate is an indolyl-based enzyme substrate.

4. The method according to claim 2 wherein the enzyme substrate is chosen from fluorescein and resorufin based enzyme substrates.

5. A method of detecting an enzyme reaction according to claim 1 comprising the steps of:
a. Obtaining a sample,
b. Contacting said sample with an enzyme substrate diagnostic material that can be used to provide for at least a fluorogenic quality and a chromogenic quality that each can be used to identify said enzyme reaction, and a different enzyme substrate diagnostic material that can be used to provide for at least a fluorogenic and chromogenic quality that each can be used to identify a different enzyme reaction, wherein at least the fluorogenic quality that can be used to detect the first said enzyme is different than the fluorogenic quality that can be used to detect the second said enzyme, and wherein the two said chromogenic quality may be the same or different,
c. Monitoring the result of step b to detect at least presence or absence of the first said fluorogenic quality representing the first said enzyme reaction,
d. Monitoring the result of step b to detect at least presence or absence of the first said chromogenic quality representing the first said enzyme reaction,
e. Monitoring the result of step b to detect at least presence or absence of the second said fluorogenic quality representing the second said enzyme reaction, and
f. Monitoring the result of step b to detect at least presence or absence of the second said chromogenic quality representing the second said enzyme reaction.

6. A method of detecting an enzyme reaction according to claim 1 comprising the steps of:
a. Obtaining a sample,
b. Contacting said sample with an enzyme substrate diagnostic material that can be used to provide for at least a fluorogenic quality and a chromogenic quality that can each be used to identify said enzyme reaction, and a different enzyme substrate diagnostic material that can be used to provide for at least a fluorogenic and chromogenic quality that can each be used to identify a different enzyme reaction, wherein at least the fluorogenic quality that can be used to detect the first said enzyme is different than the fluorogenic quality that can be used to detect the second said enzyme,
c. Monitoring the result of step b to detect at least presence or absence of the first said fluorogenic quality representing the first said enzyme reaction, and
d. Monitoring the result of step b to detect at least presence or absence of the second said fluorogenic quality representing the second said enzyme reaction.

7. A method of detecting an enzyme reaction according to claim 1, comprising the steps of:
a. Obtaining a sample,
b. Contacting said sample with an enzyme substrate diagnostic material that can be used to provide for at least a fluorogenic quality and a chromogenic quality that can each be used to identify said enzyme reaction, and
c. Monitoring the result of said contacting to detect at least presence or absence of fluorogenic quality said presence representing said enzyme reaction.

8. The method according to claim 5 wherein the enzyme substrate is an indolyl-based enzyme substrate.

9. The method according to claim 5 wherein the enzyme substrate is chosen from fluorescein and resorufin based enzyme substrates.

10. The method according to claim 6 wherein the enzyme substrate is an indolyl-based enzyme substrate.

11. The method according to claim 6 wherein the enzyme substrate is chosen from fluorescein and resorufin based enzyme substrates.

12. The method according to claim 7 wherein the enzyme substrate is an indolyl-based enzyme substrate.

13. The method according to claim 7 wherein the enzyme substrate is chosen from fluorescein and resorufin based enzyme substrates.

14. A method of detecting an enzyme reaction according to claim 1, comprising the steps of:
a. Obtaining a sample,
b. Contacting said sample with an indolyl based enzyme substrate diagnostic material that can be used to provide for at least a slow fluorogenic quality that can be used to identify said enzyme reaction, and
c. Monitoring the result of said contacting to detect at least presence or absence of slow fluorogenic quality said presence representing said enzyme reaction.

15. A method of detecting an enzyme reaction according to claim 1 comprising the steps of:
a. Obtaining a sample,
b. Contacting said sample, wherein an agar streak method is not used, with an enzyme substrate diagnostic material that can be used to provide for at least a slow fluorogenic quality that can be used to identify said enzyme reaction, and a different enzyme substrate diagnostic material that can be used to provide for at least a different slow fluorogenic quality that can be used to identify a different enzyme reaction, wherein at least the fluorogenic quality that can be used to detect the first said enzyme is different than the fluorogenic quality that can be used to detect the second said enzyme,
c. Monitoring the result of step b to detect at least presence or absence of the first said fluorogenic quality representing the first said enzyme reaction, and
d. Monitoring the result of step b to detect at least presence or absence of the second said fluorogenic quality representing the second said enzyme reaction.

16. A method of detecting an enzyme reaction according to claim 1, comprising the steps of:
a. Obtaining a sample,
b. Contacting said sample with an enzyme substrate diagnostic material that can be used to provide for at least a fast fluorogenic quality that can be used to identify said enzyme reaction, and
c. Monitoring the result of said contacting to detect at least presence or absence of fast fluorogenic quality said presence representing said enzyme reaction.

17. A method of detecting an enzyme reaction according to claim 1 comprising the steps of:
a. Obtaining a sample,
b. Contacting said sample with an enzyme substrate diagnostic material that can be used to provide for at least a fast fluorogenic quality that can be used to identify said enzyme reaction, and a different enzyme substrate diagnostic material that can be used to provide for at least a fast fluorogenic quality that can be used to identify a different enzyme reaction, wherein at least the fluorogenic quality that can be used to detect the first said enzyme is different than the fluorogenic quality that can be used to detect the second said enzyme,
c. Monitoring the result of step b to detect at least presence or absence of the first said fluorogenic quality representing the first said enzyme reaction,
d. Monitoring the result of step b to detect at least presence or absence of the second said fluorogenic quality representing the second said enzyme reaction.

18. A method of detecting a naturally fluorescent microorganism by detecting the fluorescence.

19. The method according to claim 18 wherein the microorganism is a Pseudomonas sp.

20. A method of detecting an enzyme reaction using an enzyme substrate that can be used to provide for two different detectable qualities, wherein examining timing of the first quality, and a second quality can show rate of said enzyme reaction over time.

21. A method of detecting an enzyme reaction according to claim 20, comprising the steps of:
obtaining a sample;
contacting said sample with an enzyme substrate diagnostic material that can provide for at least a fluorogenic quality and chromogenic quality representing said enzyme reaction in said sample; and
monitoring the result of said contacting to detect at least the presence or absence of chromogenic quality representing said enzyme reaction; and
monitoring the result of said contacting to detect at least the presence or absence of fluorogenic quality representing said enzyme reaction.

22. The method according to claim 21 wherein the dual enzyme substrate is an indolyl-based substrate.

23. A method of detecting an enzyme reaction according to claim 20, using an enzyme substrate diagnostic material that can be used to provide for at least two different detectable qualities, wherein at least one of these qualities is slow fluorescent if it is the only quality being monitored for.

24. The method according to claim 23 where the enzyme substrate is chosen from indolyl, resorufin, and fluorescein based substrates.

25. The method according to claim 23 wherein the enzyme substrate diagnostic material can be used to provide for at least any combination of qualities from a group comprising two or more fluorogenic and chromogenic qualities.

26. A dual enzyme substrate diagnostic material that can be used to provide for at least any combination of qualities from a group comprising two or more fluorogenic and chromogenic qualities.

27. A method according to claim 20, comprising the steps:
a. obtaining a sample;
b. contacting said sample with an enzyme substrate diagnostic material that can be used to provide for at least a quality chosen from a group comprising slow diffusing, non diffusing and precipitating fluorogenic quality for a solid test representing said enzyme reaction in said sample; and;
c. monitoring the result of step b to detect at least the presence or absence of any said fluorogenic quality representing said enzyme reaction.

28. The method according to claim 27 wherein the enzyme substrate diagnostic material has at least a quality chosen from a group comprising slow diffusing, non diffusing and precipitating fluorogenic quality

29. An enzyme substrate diagnostic material having at least a quality chosen from a group comprising slow diffusing, non diffusing and precipitating fluorogenic quality.

30. A method of claim 20, comprising the steps of:
a. obtaining a sample;
b. contacting said sample with an indolyl based enzyme substrate diagnostic material that can be used to provide for at least a quality chosen from a group comprising slow diffusing, non diffusing and precipitating fluorogenic quality representing any enzyme reaction representing the presence of any target enzyme in or produced by items in said sample; and
c. monitoring the result of said contacting to detect at least the presence or absence of any quality chosen from a group comprising slow diffusing, non diffusing and precipitating fluorogenic quality representing any said enzyme reaction.

31. A method of claim 20, comprising the steps of:
obtaining a sample;
contacting said sample with an indolyl based enzyme substrate diagnostic material that can be used to provide for at least a fluorogenic and chromogenic quality representing any enzyme reaction representing the presence of any target enzyme in or produced by items in said sample; and
monitoring the result of said contacting to detect at least the presence or absence of any chromogenic quality representing any said enzyme reaction; and
monitoring the result of said contacting to detect at least the presence or absence of any fluorogenic quality representing any said enzyme reaction.

32. A method of claim 20, comprising the steps of:
obtaining a sample;
contacting said sample with an indolyl based enzyme substrate diagnostic material that can be used to provide for at least a combination of two or more fluorogenic qualities representing enzyme reactions from at least two different substrates in said sample; and
monitoring the result of the preceding step to detect at least the presence or absence of a fluorogenic quality representing said enzyme reaction; and
monitoring the result of said preceeding step to detect at least the presence or absence of any second different fluorogenic quality representing any said enzyme reaction.

33. The method of claim 32 wherein the indolyl based enzyme substrate diagnostic material has at least any quality from a group comprising two or more fluorogenic and chromogenic qualities.

34. An enzyme indolyl based substrate diagnostic material having at least any quality from a group comprising two or more fluorogenic and chromogenic qualities.

35. The method of claim 34 wherein the enzyme indolyl based substrate diagnostic material can be used to provide at least a fluorogenic quality and a chromogenic quality.

36. An enzyme indolyl based substrate diagnostic material having at least a fluorogenic and chromogenic quality.

37. The invention according to claim 32 where a fluorescent quality dissipates, detecting said enzyme.

38. The invention according to claim 32 where a fluorescent quality that can be used to detect enzyme activity is primarily detected before a chromogenic quality that detects past enzyme activity.

39. The invention according to claim 32 where naturally fluorescent organisms are detected by their fluorescence.

40. The invention according to claim 32 where combination of 3-indolyl and N-methyl-3-indolyl based substrate provides for a more compact organism appearance.

41. A method of detecting an enzyme, comprising the steps of:
obtaining a sample;
contacting said sample with a enzyme substrate diagnostic material that can provide for at least a fast diffusing fluorogenic or chromogenic quality due to any enzyme reaction in the presence of any target enzyme in or produced by items in said sample; and
monitoring the result of said contacting to detect at least the presence or absence of any fast diffusing chromogenic quality representing any said enzyme reaction; and
monitoring the result of said contacting to detect at least the presence or absence of any fast diffusing fluorogenic quality representing any said enzyme reaction.

42. A method of detecting an enzyme, comprising the steps of:
obtaining a sample;
contacting said sample with at least two different enzyme substrate diagnostic materials that can provide for at least two contrasting fast diffusing fluorogenic or chromogenic quality due to any enzyme reaction in the presence of any one or different target enzymes in or produced by items in said sample; and
monitoring the result of said contacting to detect at least the presence or absence of any fast diffusing chromogenic quality representing any said enzyme reaction.

43. A method of claim 1 where any number of enzyme substrates can be used, which each can be used to provide for one detectable quality (i.e.—fluorescence or chromogenicity), provided that if one or more of said quality or qualities is monitored for, then there must be monitoring for at least one fluorogenic quality provided for by use of a said substrate of claim 1.

44. A method of detecting an enzyme reaction, if it occurs, using an enzyme substrate diagnostic material that can be used to provide for at least a slow fluorescent detectable quality which is monitored for detection, wherein the method is not an agar streak plate method.

45. A method of detecting an enzyme reaction, if it occurs, using an indolyl based enzyme substrate diagnostic material that can be used to provide for at least a slow fluorescent detectable quality which is monitored for detection.

46. A method of claim 1 where any number of enzyme substrates can be used, which each can be used to provide for one detectable quality (i.e.—fluorescence or chromogenicity), provided that if one or more of said quality or qualities is monitored for, then there must be monitoring for at least one fluorogenic quality provided for by use of a said substrate of the claim 44.
